(19) United States
(12) Patent Application Publication
(10) Pub. No.: US 2022/0184341 A1
(43) Pub. Date: Jun. 16, 2022

(54) TUBE STRUCTURE

(71) Applicant: COATING P. MATERIALS CO., LTD., Taichung (TW)

(72) Inventors: Cin-He CHANG, Taichung City (TW); Yung-Tai LIN, Taichung City (TW)

(21) Appl. No.: 17/228,319

(22) Filed: Apr. 12, 2021

(30) Foreign Application Priority Data
Dec. 11, 2020 (TW) 109216458

(51) Int. Cl. A61M 25/00; C08L 75/04; C08L 67/02; C08L 67/04

(52) U.S. Cl. CPC A61M 25/005; A61M 25/0023; A61M 2205/0216; C08L 67/02; C08L 67/04; C08L 75/04

(57) ABSTRACT

A tube structure includes a tube body formed by polyurethane elastomer and a wire. The tube body includes an outer surface, a first end, and a second end opposite to the first end. The outer surface is connected to the first end and the second end and the outer surface is surrounded around by the wire to form a plurality of wire gaps. A first implanting section, a connecting section, and a second implanting section are sequentially disposed from the first end to the second end of the tube body. So the plurality of wire gaps respectively form with a first wire-surrounding density, a third wire-surrounding density, and a second wire-surrounding density at the first implanting section, the connecting section and the second implanting section, wherein the first wire-surrounding density is less than the third wire-surrounding density, and the second wire-surrounding density is also less than the third wire-surrounding density.

### FIELD OF THE INVENTION

The present invention relates to a tube structure, more particularly to a tube structure is capable of kink and puncture resistance simultaneously.

### BACKGROUND OF THE INVENTION

Medical catheters, such as vascular prosthesis, are substitutions for thrombosis or angiostenosis of native blood vessels. The medical catheters are made by nylon in early days and polytetrafluoroethylene (PTFE) presently, where expanded PTFE (ePTFE) is more common. The main manufacturing method of the ePTFE catheter is expanded-elongation method, and presently the ePTFE catheters have been applied on therapies of diseases on artery or vein.

The medical catheters always need to engage a plurality of obstacles such as other tissues, blood vessels or organs near the affected region when the medical catheters are implanted or applied into the human body or other experimental bodies. However, these traditional medical catheters do not possess good kink resistance. The traditional medical catheter can be easily kinked or folded by squeezing, and the liquid flowed inside the medical catheter after implanting into the human body can also be easily affected. On the contrary, the complication such as thrombosis may be formed due to the emphraxis inside the medical catheter.

In addition, the medical catheters are usually applied on some treating processes such as hemodialysis due to renal failure. However, as the traditional medical catheters do not possess good puncture resistance, the bleeding may easily occur when the injection time increases on the medical catheter. It may need to implant another medical catheter if the bleeding is not hemostatic. Thus, it is still needed to provide a medical catheter with both kink and puncture resistances.

### SUMMARY OF THE INVENTION

According to above drawbacks, an object of the invention is to provide a tube structure to improve kink resistance even if the tube structure is folded.

It is another object of the invention is to provide a tube structure to increase the puncture resistance of the tube body to reduce the internal liquid leakage out of the tube structure due to the tube structure is punctured.

In order to achieve above objects, the invention provides a tube structure which includes a tube body and a wire surrounded around the outer surface of the tube body, the material of the tube body is polyurethane elastomer. The tube body includes an outer surface, a first end and, a second end opposite to the first end, where the outer surface is located between the first end and the second end, and the outer surface is connected to the first end and the second end. The wire is surrounded around the outer surface of the tube body to form a plurality of wire gaps on the outer surface of the tube body. A first implanting section, a connecting section and a second implanting section are disposed sequentially on the outer surface of the tube body, the connecting section is located between the first implanting section and the second implanting section, so the plurality of wire gaps respectively form a first wiring density, a third wiring density and a second wiring density formed on the first implanting section, the connecting section and the second implanting section, in which the first wiring density is less than the third wiring density and the second wiring density is less than the third wiring density.

In another embodiment, the wire is spirally surrounded around on the outer surface of the tube body.

In another embodiment, the first wiring density is equal to the second wiring density.

In another embodiment, the material of the wire is polybutylene terephthalate.

In another embodiment, the thickness of the wire is between 0.2 to 3 mm on the outer surface.

In another embodiment, a protecting layer simultaneously covers the wires around the outer surface of the tube body and the outer surface of the tube body.

In another embodiment, the material of the protecting layer is polyurethane or polylactic acid.

In another embodiment, the wire gaps are between 1 to 100 mm.

In another embodiment, the wire is integrally formed surrounded around the outer surface.

In another embodiment, an inner diameter of the tube body is between 1 to 100 mm.

According to the above description, the wire is surrounded around the outer surface of the tube body to form a plurality of wire gaps on the outer surface. The plurality of wire gaps form with the first wiring density, the third wiring density, and the second wiring density on the first implanting section, the connecting section and the second implanting section, in which the first wiring density is less than the third wiring density, and the second wiring density is less than the third wiring density. Thus, the tube structure possesses excellent kink resistance to decrease the existence of kink phenomenon when the tube structure is folded, and the tube structure also possesses excellent puncture resistance to reduce the internal liquid leakage out of the tube body due to the tube structure is punctured.

### BRIEF DESCRIPTION OF THE DRAWINGS

FIG. 1 is a schematic view of the tube structure in accordance with one embodiment of the invention;

FIG. 2 is a cross-section view of region A in FIG. 1 in accordance with one embodiment of the invention; and

FIG. 3 is a schematic view of the tube structure in accordance with another embodiment of the invention.

### DETAILED DESCRIPTION OF THE PREFERRED EMBODIMENT

The advantages and characteristics of the invention and the way to achieve the purpose of the invention will be easily understood by referring to the exemplary embodiments and the drawings. However, the invention can be embodied by different forms and should not be understood that the embodiments herein are limited to the invention. On the contrary, for persons ordinarily skilled in the art, the provided embodiments will express the scope of the present invention more thoroughly, more wholly and more completely.

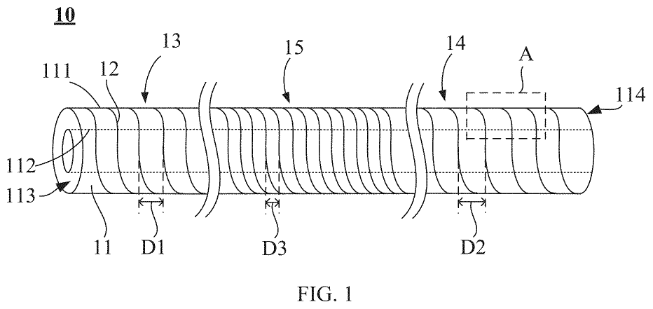

FIG. 1 is a schematic view of the tube structure in accordance with one embodiment of the invention. Please refer to FIG. 1. The tube structure 10 includes a tube body 11 and a wire 12, where the tube body 11 is formed by polyurethane (PU) elastomer and can be use as vascular prosthesis. The tube body 11 includes an outer surface 111, an inner surface 112, a first end 113, and a second end 114 opposite to the first end 113. The outer surface 111 is opposite to the inner surface 112. The outer surface 111 is located between the first end 113 and the second end 114, and the outer surface is connected to the first end 113 and second end 114. A first implanting section 13, a connecting section 15 and a second implanting section 14 are disposed sequentially on the outer surface 111 from the first end 113 to the second end 114, and the connecting section 15 is located between the first implanting section 13 and the second implanting section 14. In the embodiment, the first implanting section 13 and the second implanting section 15 are used to engage with the existed blood vessels when the tube structure 10 is implanted into organism such as human bodies.

The wire 12 is surrounded around the outer surface 111 of the tube body 11 to form a plurality of wire gaps D1, D2 and D3 on the outer surface 111, in which a plurality of wire gaps respectively form with a first wiring density d1 on the first implanting section 13, a third wiring density d3 on the connecting section 15, and a second wiring density d2 on the second implanting section 14. For example, the wiring density d1, d2 or d3 can be the amount of wire gaps D1, D2 or D3 in predetermined length on the outer surface 111 of the tube body 11, or number of winds of the wire 12. In the embodiment, the first wiring density d1 is less than the third wiring density d3, and the second wiring density d2 is less than the third wiring density d3 (d1, d2 and d3 are not shown in FIG. 1). In other words, the wire gap D1 of the first implanting section 13 and the wire gap D2 of the second implanting section 14 are both larger than the wire gap D3 of the connecting section 15. Thus, the connecting section 15 possesses excellent kink resistance due to higher wiring density d3, and the first implanting section 13 and the second implanting section 15 possess better flexibility due to lower wiring densities d1 and d2. The hardness of engaging to the existed blood vessels of the human body can be decreased when the tube structure 10 is implanted to the human body. The wire 12 is integrally formed to surround around the outer surface 111 to decrease the processing cost of the wire 12, however, the wire 12 can also be formed separately on different sections of the outer surface 111. In addition, the first wiring density d1 can be equal to the second density d2 to decrease the difficulty of designation and manufacturing of the tube structure 10.

In the embodiment, the material of the wire 12 is polybutylene terephthalate (PBT), so the wire surrounded around the outer surface of the tube structure 10 is not only to increase the mechanical strength of the tube structure 10 but also increase the biocompatibility of the tube structure 10 made by PBT. The material of the tube body 11 is PU elastomer, where PU elastomer has better mechanical property than ePTFE such as elasticity and toughness, thus, the puncture resistance of the tube body 11 can be increased. Even if the tube body 11 is impaled by external force to cause the impaled hole on the tube body 11, by the elasticity of PU elastomer, the outer surface of the tube body 11 can be restored to let the outer surface without any impaled hole thereon, so that the internal liquid leakages out of the tube body 11 can be largely reduced. Therefore, the designation complexity of the tube body 11 can be decreased, and a special puncture resistance layer is not necessary to dispose on the outer surface 111 or the inner surface 112.

Table 1 is a comparison table of a bleeding experiment between the tube body 11 of the embodiment and a traditional ePTFE medical catheter (hereinafter, ePTFE catheter), where the wall thickness, the inner diameter, size of the puncturing needle and the puncturing time between the tube body 11 and the ePTFE catheter are the same. According to table 1, the bleeding volume of the tube body 11 formed by PU elastomer is nearly one thousandth of the ePTFE catheter.

TABLE 1

| tube body/catheter type | PU | ePTFE |
|---|---|---|
| bleeding volume (mg) | 2.1 ± 1.2 | 3121.0 ± 1665.0 |

The tube structure 10 possesses kink resistance and biocompatibility from PBT and puncturing resistance from PU elastomer simultaneously due to the tube body 11 and the wire 12 surrounded around the outer surface 111. The thickness of the wire 12 on the outer surface 111 is between 0.2 to 3 mm. The forming method of the wire 12 is, for example, heating the raw materials of PBT to melting state and then spreading the melting PBT onto the tube body 11 by an injector with small caliber. In addition, the wire gaps D1, D2 or D3 are between 1 to 100 mm and the suitable inner diameter of the tube body 11 is between 1 to 100 mm, therefore, the kink resistance of the tube structure 10 can be upgraded significantly. Furthermore, the tube body 11 is formed by one PU elastomer layer in the embodiment, however, the tube body 11 may also formed by two or more PU elastomer layers to possess more excellent puncture resistance.

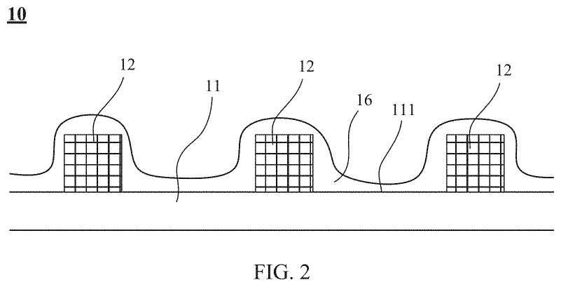

FIG. 2 is a cross-section view of region A in accordance with FIG. 1. Please refer to FIGS. 1 and 2, the tube structure 10 further selectively includes a protecting layer 16 to cover the wire 12 around the outer surface 111 and the outer surface 111 of the tube body 11 simultaneously. The wire 12 can be fixed on a predetermined position of the outer surface 111, and the wire 12 can be protected from extruding or dropping off from the predetermined position of the outer surface 111 by external force or obstacles when the tube structure 10 is implanted into the human body. The material of the protecting layer 16 can be PU or polylactic acid (PLA), and the protecting layer 16 can be manufactured on the outer layer 111 and the wire 12 by spinning and depositing method. The protecting layer 16 can also be formed by PU and PLA simultaneously to enhance the biocompatibility of the tube structure 10. In addition, the surrounding position of the wire 12 of the tube structure 10 can be changed by manual or forceps to easily peel off the wire 12 and the protecting layer 16 from the outer surface 111. Thus, the agility of kink resistance can be easily modified better than the traditional medical catheter manufactured by expanding and stretching method.

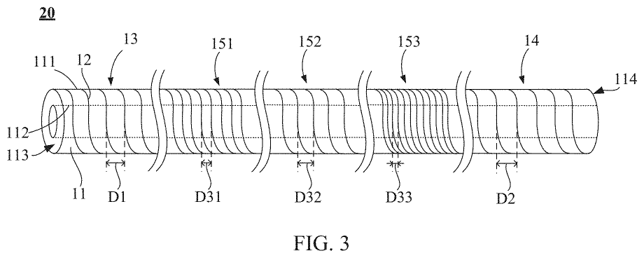

FIG. 3 is a schematic view of the tube structure in accordance with another embodiment of the invention. Please refer to FIG. 3, the tube structure 20 is similar to the tube structure 10 in the above-mentioned embodiment, where the same elements are indicated by the same numbers. The difference between the tube structure 10 and the tube structure 20 is that the outer surface 111 of the tube body 11 further includes a plurality connecting sections (FIG. 3 shows three connecting sections 151, 152 and 153 but the invention is not limited herein). The wire gaps D31 with wiring densities d31, the wire gaps D32 with wiring densities d32, and the wire gaps D33 with wiring densities d33 are in the connecting sections 151, 152 and 153 respectively. The wiring densities d31, d32 and d33 are different with each other, where the average wiring density d30 of the connecting sections 151, 152 and 153 is higher than the first wiring density d1 of the first implanting section 13 and the second wiring density d2 of the second implanting section 14. Thus, each wiring density of the connecting section can be respectively modified when the tube structure 20 is implanted into different parts of the human body, and each connecting section can possess different degree of kink resistance to engage the tissues, obstacles or organs in the implanted part of the human body.

In summary of the above, the wire is surrounded around the outer surface of the tube body to form a plurality of wire gaps. The plurality of wire gaps form with the first wire density, the third wire density and the second wire density on the first implanting section, the connecting section, and the second implanting section in which the first wiring density is less than the third wiring density, and the second wiring density is less than the third wiring density. Thus, the tube structure possesses excellent kink resistance even if the tube structure is folded. The tube structure also possesses excellent puncture resistance to reduce the internal liquid leakage out of the tube body due to the tube structure is punctured.

The above is only the preferred embodiment of the invention, and the scope of the invention is not limited thereto, that is, the simple equivalent changes and modifications made by the scope of the invention and the description of the invention are all remain within the scope of the invention. Furthermore, terms, such as “first,” “second,” etc., mentioned in the specification or claims are simply for naming the elements or distinguishing different embodiments or scopes, and thus should not be construed as the upper or lower limit of the number of any element.

What is claimed is:

1. A tube structure, comprising:
a tube body formed by polyurethane elastomer includes an outer surface, a first end, and a second end opposite to the first end, where the outer surface is located between the first end and the second end, and the outer surface is connected to the first end and the second end; and
a wire is surrounded around the outer surface of the tube body to form a plurality of wire gaps is on the outer surface;
wherein a first implanting section, a connecting section and a second implanting section are disposed sequentially on the outer surface from the first end to the second end of the tube body, the connecting section is located between the first implanting section and the second implanting section, so the plurality of wire gaps respectively form with a first wiring density, a third wiring density and a second wiring density at the first implanting section, the connecting section, and the second implanting section, wherein the first wiring density is less than the third wiring density and the second wiring density is less than the third wiring density.

2. The tube structure of claim 1, wherein the wire is spirally disposed around on the outer surface of the tube body.

3. The tube structure of claim 1, wherein the first wiring density is equal to the second wiring density.

4. The tube structure of claim 1, wherein the material of the wire is polybutylene terephthalate.

5. The tube structure of claim 1, wherein a thickness of the wire is between 0.2 to 3 mm on the outer surface.

6. The tube structure of claim 1, further comprising a protecting layer simultaneously covers the wire around the outer surface of tube body and the outer surface of the tube body.

7. The tube structure of claim 6, wherein the material of the protecting layer is polyurethane or polylactic acid.

8. The tube structure of claim 1, wherein the wire gaps are between 1 to 100 mm.

9. The tube structure of claim 1, wherein the wire is integrally formed to surround around the outer surface of the tube body.

10. The tube structure of claim 1, wherein an inner diameter of the tube body is between 1 to 100 mm.
